(19) United States
(12) Patent Application Publication
(10) Pub. No.: US 2012/0225097 A1
(43) Pub. Date: Sep. 6, 2012

(54) PIPERAZINECARBOXAMIDE DERIVATIVE USEFUL AS A MODULATOR OF FATTY ACID AMIDE HYDROLASE (FAAH)

(76) Inventor: Natalie A. Hawryluk, San Diego, CA (US)

(21) Appl. No.: 13/508,975

(22) PCT Filed: Nov. 10, 2010
(86) PCT No.: PCT/US10/56166

§ 371 Date: May 9, 2012

Related U.S. Application Data
(60) Provisional application No. 61/260,827, filed on Nov. 12, 2009.
(60) Provisional application No. 61/263,477, filed on Nov. 23, 2009.

(51) Int. Cl. A61K 39/00; A61K 31/496; A61P 25/22; A61P 25/24; A61P 25/20; A61P 29/00; A61P 3/04; A61P 31/18; A61P 25/00; A61P 25/28; A61P 25/08; A61P 25/16; A61P 27/02; A61P 37/00; A61P 25/30; A61P 15/00; A61P 1/00; A61P 37/06; A61P 1/12; A61P 1/04; A61P 19/02; A61P 9/12; A61P 35/00; A61P 37/08; A61P 3/10; A61P 3/00; A61P 19/10; C07D 401/12

(52) U.S. Cl. 424/278.1; 544/360; 514/253.01

(57) ABSTRACT

N-Pyridin-3-yl-4-{3-[4-(trifluoromethyl)phenoxy]benzyl}piperazine-1-carboxamide is described, which is useful as a FAAH inhibitor. N-Pyridin-3-yl-4-{3-[4-(trifluoromethyl)phenoxy]benzyl}piperazine-1-carboxamide may be used in pharmaceutical compositions and methods for the treatment of disease states, disorders, and conditions mediated by fatty acid amide hydrolase (FAAH) activity, such as anxiety, pain, inflammation, sleep disorders, eating disorders, energy metabolism disorders, and movement disorders (e.g., multiple sclerosis). A method of synthesizing N-pyridin-3-yl-4-{3-[4-(trifluoromethyl)phenoxy]benzyl}piperazine-1-carboxamide is also disclosed.

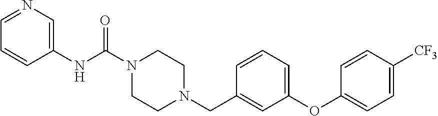

### CROSS REFERENCE TO RELATED APPLICATIONS

This application claims the benefit of U.S. provisional patent application Ser. No. 61/260,827, filed on Nov. 12, 2009 and U.S. provisional patent application Ser. No. 61/263,477, filed on Nov. 23, 2009

### FIELD OF THE INVENTION

The present invention relates to the compound of N-pyridin-3-yl-4-{3-[4-(trifluoromethyl)phenoxy]benzyl}piperazine-1-carboxamide, pharmaceutical compositions containing the compound, and methods of using the compound for the treatment of disease states, disorders, and conditions mediated by fatty acid amide hydrolase (FAAH) activity are provided.

### BACKGROUND OF THE INVENTION

Medicinal benefits have been attributed to the cannabis plant for centuries. The primary bioactive constituent of cannabis is Δ⁹-tetrahydrocannabinol (THC). The discovery of THC eventually led to the identification of two endogenous cannabinoid receptors responsible for its pharmacological actions, namely CB₁ and CB₂ (Goya, Exp. Opin. Ther. Patents 2000, 10, 1529). These discoveries not only established the site of action of THC, but also inspired inquiries into the endogenous agonists of these receptors, or “endocannabinoids”. The first endocannabinoid identified was the fatty acid amide anandamide (AEA). AEA itself elicits many of the pharmacological effects of exogenous cannabinoids (Piomelli, Nat. Rev. Neurosci. 2003, 4(11), 873).

The catabolism of AEA is primarily attributable to the integral membrane bound protein fatty acid amide hydrolase (FAAH), which hydrolyzes AEA to arachidonic acid. FAAH was characterized in 1996 by Cravatt and co-workers (Cravatt, Nature 1996, 384, 83). It was subsequently determined that FAAH is additionally responsible for the catabolism of a large number of important lipid signaling fatty acid amides including: another major endocannabinoid, 2-arachidonoylglycerol (2-AG) (Science 1992, 258, 1946-1949); the sleep-inducing substance, oleamide (OEA) (Science 1995, 268, 1506); the appetite-suppressing agent, N-oleoylethanolamine (Rodriguez de Fonesca, Nature 2001, 414, 209); and the anti-inflammatory agent, palmitoylethanolamide (PEA) (Lambert, Curr. Med. Chem. 2002, 9(6), 663).

Small-molecule inhibitors of FAAH should elevate the concentrations of these endogenous signaling lipids and thereby produce their associated beneficial pharmacological effects. There have been some reports of the effects of various FAAH inhibitors in pre-clinical models.

In particular, two carbamate-based inhibitors of FAAH were reported to have analgesic properties in animal models. In rats, BMS-1 (see WO 02/087569), which has the structure shown below, was reported to have an analgesic effect in the Chung spinal nerve ligation model of neuropathic pain, and the Hargraves test of acute thermal nociception. URB-597 was reported to have efficacy in the zero plus maze model of anxiety in rats, as well as analgesic efficacy in the rat hot plate and formalin tests (Kathuria, Nat. Med. 2003, 9(1), 76). The sulfonylfluoride AM374 was also shown to significantly reduce spasticity in chronic relapsing experimental autoimmune encephalomyelitis (CREAE) mice, an animal model of multiple sclerosis (Baker, FASEB J. 2001, 15(2), 300).

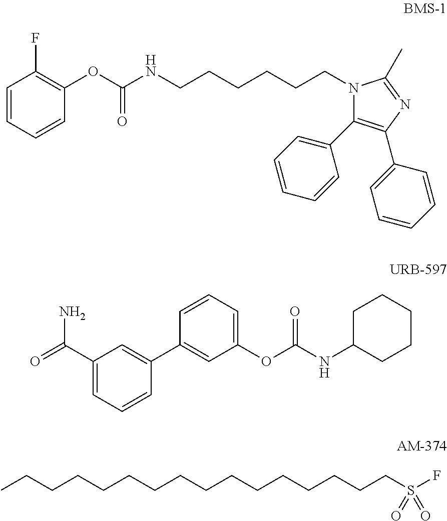

In addition, the oxazolopyridine ketone OL-135 is reported to be a potent inhibitor of FAAH, and has been reported to have analgesic activity in both the hot plate and tail emersion tests of thermal nociception in rats (WO 04/033652).

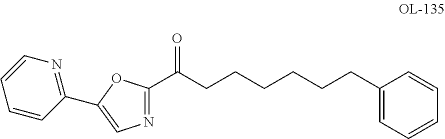

Results of research on the effects of certain exogenous cannabinoids has elucidated that a FAAH inhibitor may be useful for treating various conditions, diseases, disorders, or symptoms. These include pain, nausea/emesis, anorexia, spasticity, movement disorders, epilepsy and glaucoma. To date, approved therapeutic uses for cannabinoids include the relief of chemotherapy-induced nausea and emesis among patients with cancer and appetite enhancement in patients with HIV/AIDs who experience anorexia as a result of wasting syndrome. Two products are commercially available in some countries for these indications, namely, dronabinol (Marinol®) and nabilone.

Apart from the approved indications, a therapeutic field that has received much attention for cannabinoid use is analgesia, i.e., the treatment of pain. Five small randomized controlled trials showed that THC is superior to placebo, producing dose-related analgesia (Robson, Br. J. Psychiatry 2001, 178, 107-115). Atlantic Pharmaceuticals is reported to be developing a synthetic cannabinoid, CT-3, a 1,1-dimethyl heptyl derivative of the carboxylic metabolite of tetrahydrocannabinol, as an orally active analgesic and anti-inflammatory agent. A pilot phase II trial in chronic neuropathic pain with CT-3 was reportedly initiated in Germany in May 2002.

A number of individuals with locomotor activity-related diseases, such as multiple sclerosis have claimed a benefit from cannabis for both disease-related pain and spasticity, with support from small controlled trials (Croxford et al., J. Neuroimmunol, 2008, 193, 120-9; Svendsen, Br. Med. J. 2004, 329, 253). Likewise, various victims of spinal cord injuries, such as paraplegia, have reported that their painful spasms are alleviated after smoking marijuana. A report showing that cannabinoids appear to control spasticity and tremor in the CREAE model of multiple sclerosis demonstrated that these effects are mediated by CB₁ and CB₂ receptors (Baker, Nature 2000, 404, 84-87). Phase 3 clinical trials have been undertaken in multiple sclerosis and spinal cord injury patients with a narrow ratio mixture of tetrahydrocannabinol/cannabidiol (THC/CBD). It has been reported that FAAH knockout mice consistently recover to a better clinical score than wild type controls, and this improvement is not a result of anti-inflammatory activity, but rather may reflect some neuroprotection or remyelination promoting effect of lack of the enzyme (Webb et al, Neurosci Lett., 2008, vol. 439, 106-110).

Reports of small-scale controlled trials to investigate other potential commercial uses of cannabinoids have been made. Trials in volunteers have been reported to have confirmed that oral, injected, and smoked cannabinoids produced dose-related reductions in intraocular pressure (10P) and therefore may relieve glaucoma symptoms. Ophthalmologists have prescribed cannabis for patients with glaucoma in whom other drugs have failed to adequately control intraocular pressure (Robson, 2001, supra).

Inhibition of FAAH using a small-molecule inhibitor may be advantageous compared to treatment with a direct-acting CB₁ agonist. Administration of exogenous CB₁ agonists may produce a range of responses, including reduced nociception, catalepsy, hypothermia, and increased feeding behavior. These four in particular are termed the “cannabinoid tetrad.” Experiments with FAAH −/− mice show reduced responses in tests of nociception, but did not show catalepsy, hypothermia, or increased feeding behavior (Cravatt, Proc. Natl. Acad. Sci. USA 2001, 98(16), 9371). Fasting caused levels of AEA to increase in rat limbic forebrain, but not in other brain areas, providing evidence that stimulation of AEA biosynthesis may be anatomically regionalized to targeted CNS pathways (Kirkham, Br. J. Pharmacol. 2002, 136, 550). The finding that AEA increases are localized within the brain, rather than systemic, suggests that FAAH inhibition with a small molecule could enhance the actions of AEA and other fatty acid amides in tissue regions where synthesis and release of these signaling molecules is occurring in a given pathophysiological condition (Piomelli, 2003, supra).

In addition to the effects of a FAAH inhibitor on AEA and other endocannabinoids, inhibitors of FAAH's catabolism of other lipid mediators may be used in treating certain other therapeutic indications. For example, PEA has demonstrated biological effects in animal models of inflammation (Holt, et al. Br. J. Pharmacol. 2005, 146, 467-476), immunosuppression, analgesia, and neuroprotection (Ueda, J. Biol. Chem. 2001, 276(38), 35552). Oleamide, another substrate of FAAH, induces sleep (Boger, Proc. Natl. Acad. Sci. USA 2000, 97(10), 5044; Mendelson, Neuropsychopharmacology 2001, 25, S36). Inhibition of FAAH has also been implicated in cognition (Varvel et al., J. Pharmacol. Exp. Ther. 2006, 317(1), 251-257) and depression (Gobbi et al., PNAS, USA 2005, 102(51), 18620-18625).

Two additional indications for FAAH are supported by recent data indicating that FAAH substrate activated receptors are important in energy metabolism, and in bone homeostasis (Overton et al., Br. J. Pharmacol. 2008, in press; and Plutzky, Diab. Vasc. Dis. Res. 2007, 4 Suppl 3, S12-4). It has been shown that the previously mentioned lipid signaling fatty acid amides catabolized by FAAH, oleoylethanolamide (OEA), is one of the most active agonists of the recently de-orphanised GPCR 119 (GPR119) (also termed glucose dependent insulinotropic receptor). This receptor is expressed predominantly in the pancreas in humans and activation improves glucose homeostasis via glucose-dependent insulin release in pancreatic beta-cells. GPR119 agonists can suppress glucose excursions when administered during oral glucose tolerance tests, and OEA has also been shown independently to regulate food intake and body weight gain when administered to rodents, indicating a probable benefit energy metabolism disorders, such as insulin resistance and diabetes. The FAAH substrate palmitoylethanolamide (PEA) is an agonist at the PPARα receptor. Evidence from surrogate markers in human studies with the PPARα agonist fenofibrate is supportive of the concept that PPARα agonism offers the potential for inducing a coordinated PPARα response that may improve dyslipidaemia, repress inflammation and limit atherosclerosis in patients with the metabolic syndrome or type 2 diabetes. The FAAH substrate anandamide (AEA) is an agonist at the PPARγ receptor. Anandamide treatment induces 3T3-L1 differentiation into adipocytes, as well as triglyceride droplet accumulation and expression of adiponectin (Bouaboula et al., E. J. Pharmacol. 2005, 517, 174-181). Low dose cannabinoid therapy has been shown to reduce atherosclerosis in mice, further suggesting a therapeutic benefit of FAAH inhibition in dyslipidemia, liver steatosis, steatohepatitis, obesity, and metabolic syndrome (Steffens et al., Nature, 2005, 434, 782-6).

Osteoporosis is one of the most common degenerative diseases. It is characterized by reduced bone mineral density (BMD) with an increased risk for bone fractures. CB₂-deficient mice have a markedly accelerated age-related trabecular bone loss and cortical expansion. A CB₂-selective agonism enhances endocortical osteoblast number and activity and restrains trabecular osteoclastogenesis and attenuates ovariectomy-induced bone loss (Ofek et al., Proc. Natl. Acad. Sci. U.S.A. 2006, 103, 696-701). There is a substantial genetic contribution to BMD, although the genetic factors involved in the pathogenesis of human osteoporosis are largely unknown. The applicability to human BMD is suggested by genetic studies in which a significant association of single polymorphisms and haplotypes was found encompassing the CNR2 gene on human chromosome 1 p36, demonstrating a role for the peripherally expressed CB₂ receptor in the etiology of osteoporosis (Karsak et al., Hum. Mol. Genet, 2005, 14, 3389-96). Research also demonstrates a role in osteoarthritis.

Thus, small-molecule FAAH inhibitors should be useful in treating pain of various etiologies, anxiety, multiple sclerosis, Parkinson's disease and other movement disorders, nausea/emesis, eating disorders, epilepsy, glaucoma, inflammation, itch, immunosuppression, neuroprotection, depression, cognition enhancement, sleep disorders, dyslipidemia, liver steatosis, steatohepatitis, obesity, metabolic syndrome, osteoporosis, and other diseases/disorders referenced above, and potentially with fewer side effects than treatment with an exogenous cannabinoid.

Certain piperazinyl or piperidinyl derivatives have been disclosed in the literature for different uses. For example, JP 11139969 describes certain phenol derivatives as antioxidants and ACAT inhibitors; WO 96/21648 discloses various piperazine derivatives as antitumor agents; JP 48010160 describes certain piperazine derivatives as anti-inflammatory agents; WO 04/072025 discloses certain substituted N-arylheterocycles as obesity, diabetes, and drug abuse agents; DE 2123784 and U.S. Pat. No. 3,813,395 disclose various piperazinylthieno-benzothiazepines as psychotropics and anesthetics; and WO 98/37077 and WO 99/42107 describe certain piperazine-based compounds as calcitonin mimetics for treatment of bone deficits. Additionally, WO 97/42230 describes a solid-phase synthesis of certain piperazine ureas. WO 97/23458 discloses certain piperidine derivatives as intermediates toward NMDA receptor ligands.

Various small-molecule FAAH modulators have been reported, e.g., in WO 04/033652, U.S. Pat. No. 6,462,054, U.S. Pat. No. 6,096,784, WO 99/26584, WO 97/49667, and WO 96/09817. Furthermore, U.S. Pat. No. WO 2006/074025 and U.S. patent application Ser. No. 12/557,650 disclose certain piperazinyl or piperidinyl derivatives as FAAH modulators. Certain urea compounds having a piperidine or piperazine ring, which are useful as FAAH inhibitors, are disclosed in WO 08/023720. However, despite the progress that has been achieved, there remains a desire for potent FAAH modulators with suitable pharmaceutical properties.

The features and advantages of the present invention are apparent to one of ordinary skill in the art. Based upon this disclosure, including the summary, detailed description, background, examples, and claims, one of ordinary skill in the art will be able to make modifications and adaptations to various conditions and usages. Publications described herein are incorporated by reference in their entirety.

### SUMMARY OF THE INVENTION

N-Pyridin-3-yl-4-{3-[4-(trifluoromethyl)phenoxy]benzyl}piperazine-1-carboxamide, and pharmaceutically acceptable salts thereof, are herein described, which have been found to have FAAH-modulating activity. The invention is directed to the general and preferred embodiments defined, respectively, and by the independent and dependent claims appended hereto, which are incorporated by reference herein.

The present invention provides experimental evidence demonstrating that the chemical entities of the present invention exhibit unexpected and surprising cardio-toxicity profiles, minimize cardiac damage, and minimize reduction in heart rate. The chemical entities of the present invention display potency for inhibition of anandamide hydrolysis by human and rat FAAH as determined in cellular homogenate assays. These potencies are improved in comparison to previously described piperazinyl urea compounds (see U.S. Pat. No. 7,598,249). Furthermore, the chemical entities of the present invention display unpredicted characteristics in a Langendorff isolated heart assay when compared to similar molecular structures. In particular, comparator compound 4-({3-[(4-chlorophenyl)oxy]phenyl}methyl)-N-pyridin-3-ylpiperazine-1-carboxamide caused an increase in left ventricular systolic pressure in such an assay while N-pyridin-3-yl-4-{3-[4-(trifluoromethyl)phenoxy]benzyl}piperazine-1-carboxamide did not. Also of interest, addition of 4-({3-[(4-chlorophenyl)oxy]phenyl}methyl)-N-pyridin-3-ylpiperazine-1-carboxamide resulted in a near-doubling of left ventricular developed pressure (LVDevP) whereas N-pyridin-3-yl-4-{3-[4-(trifluoromethyl)phenoxy]benzyl}piperazine-1-carboxamide resulted in a decrease in LVDevP. Additionally, both comparator compounds, 4-({3-[(4-chlorophenyl)oxy]phenyl}methyl)-N-pyridin-3-ylpiperazine-1-carboxamide and 4-[3-(4-bromophenoxy)benzyl]-N-pyridin-3-ylpiperazine-1-carboxamide, caused a greater than 30% reduction in heart rate (4/4 for chlorophenyl and 3/4 for bromophenyl moieties) in rat specimens tested. In contrast, N-pyridin-3-yl-4-{3-[4-(trifluoromethyl)phenoxy]benzyl}piperazine-1-carboxamide exhibited a 30% reduction in heart rate in two of five rats tested. Moreover, in rat dosing experiments, rats treated with comparator compound 4-({3-[(4-chlorophenyl)oxy]phenyl}methyl)-N-pyridin-3-ylpiperazine-1-carboxamide developed lesions in their heart muscle, while rats treated with N-pyridin-3-yl-4-{3-[4-(trifluoromethyl)phenoxy]benzyl}piperazine-1-carboxamide lacked such lesions.

In one general aspect, the invention is directed to the compound of N-pyridin-3-yl-4-{3-[4-(trifluoromethyl)phenoxy]benzyl}piperazine-1-carboxamide. In a particular embodiment, the compound is a hydrochloride salt of N-pyridin-3-yl-4-{3-[4-(trifluoromethyl)phenoxy]benzyl}piperazine-1-carboxamide. In a further embodiment, the compound is N-pyridin-3-yl-4-{3-[4-(trifluoromethyl)phenoxy]benzyl}piperazine-1-carboxamide bis-hydrochloride dihydrate.

The invention also relates to pharmaceutically acceptable salts of N-pyridin-3-yl-4-{3-[4-(trifluoromethyl)phenoxy]benzyl}piperazine-1-carboxamide, pharmaceutically acceptable prodrugs of N-pyridin-3-yl-4-{3-[4-(trifluoromethyl)phenoxy]benzyl}piperazine-1-carboxamide, and pharmaceutically acceptable metabolites of N-pyridin-3-yl-4-{3-[4-(trifluoromethyl)phenoxy]benzyl}piperazine-1-carboxamide.

In a further general aspect, the invention relates to pharmaceutical compositions each comprising: (a) a therapeutically effective amount of at least one of N-pyridin-3-yl-4-{3-[4-(trifluoromethyl)phenoxy]benzyl}piperazine-1-carboxamide, pharmaceutically acceptable salts of N-pyridin-3-yl-4-{3-[4-(trifluoromethyl)phenoxy]benzyl}piperazine-1-carboxamide, pharmaceutically acceptable prodrugs of N-pyridin-3-yl-4-{3-[4-(trifluoromethyl)phenoxy]benzyl}piperazine-1-carboxamide, and pharmaceutically acceptable metabolites of N-pyridin-3-yl-4-{3-[4-(trifluoromethyl)phenoxy]benzyl}piperazine-1-carboxamide; and (b) a pharmaceutically acceptable excipient.

In another aspect, embodiments of the invention are useful as FAAH modulators. Thus, the invention is directed to a method for modulating FAAH activity, comprising exposing FAAH to a therapeutically effective amount of at least one of N-pyridin-3-yl-4-{3-[4-(trifluoromethyl)phenoxy]benzyl}piperazine-1-carboxamide, pharmaceutically acceptable salts of N-pyridin-3-yl-4-{3-[4-(trifluoromethyl)phenoxy]benzyl}piperazine-1-carboxamide, pharmaceutically acceptable prodrugs of N-pyridin-3-yl-4-{3-[4-(trifluoromethyl)phenoxy]benzyl}piperazine-1-carboxamide, and pharmaceutically active metabolites of N-pyridin-3-yl-4-{3-[4-(trifluoromethyl)phenoxy]benzyl}piperazine-1-carboxamide.

In another general aspect, the invention is directed to a method of treating a subject suffering from or diagnosed with a disease, disorder, or medical condition mediated by FAAH activity, comprising administering to the subject in need of such treatment an effective amount of at least one agent selected from N-pyridin-3-yl-4-{3-[4-(trifluoromethyl)phenoxy]benzyl}piperazine-1-carboxamide and its pharmaceutically acceptable salts, pharmaceutically active prodrugs, and pharmaceutically active metabolites. In preferred embodiments of the inventive method, the disease, disorder, or medical condition is selected from: anxiety, depression, pain, sleep disorders, eating disorders, inflammation, multiple sclerosis and other movement disorders, HIV wasting syndrome, closed head injury, stroke, learning and memory disorders, Alzheimer's disease, epilepsy, Tourette's syndrome, Niemann-Pick disease, Parkinson's disease, Huntington's chorea, optic neuritis, autoimmune uveitis, symptoms of drug or alcohol withdrawal, nausea, emesis, sexual dysfunction, anxiety, post-traumatic stress disorder, cerebral vasospasm, glaucoma, irritable bowel syndrome, inflammatory bowel disease, immunosuppression, itch, gastroesophageal reflux disease, paralytic ileus, secretory diarrhea, gastric ulcer, rheumatoid arthritis, unwanted pregnancy, hypertension, cancer, hepatitis, allergic airway disease, auto-immune diabetes, intractable pruritis, neuroinflammation, diabetes, metabolic syndrome, osteoporosis, dyslipidemia, liver steatosis, and steatohepatitis.

Additional embodiments, features, and advantages of the invention will be apparent from the following detailed description and through practice of the invention.

### DETAILED DESCRIPTION OF THE INVENTION

The invention may be more fully appreciated by reference to the following description, including the following glossary of terms and the concluding examples. For the sake of brevity, the disclosures of the publications cited in this specification are herein incorporated by reference.

As used herein, the terms “including”, “containing” and “comprising” are used herein in their open, non-limiting sense.

A structural formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, or fluorine, such as ²H, ³H, ¹¹C, ¹³C, ¹⁵N, ¹⁸O, ¹⁷O, and ¹⁸F, respectively. Such isotopically labeled compounds are useful in metabolic studies (preferably with ¹⁴C), reaction kinetic studies (with, for example ²H or ³H), detection or imaging techniques [such as positron emission tomography (PET) or single-photon emission computed tomography (SPECT), including drug or substrate tissue distribution assays, or in radioactive treatment of patients. In particular, an ¹⁸F- or ¹¹C-labeled compound may be preferred for PET or SPECT studies. Further, substitution with heavier isotopes such as deuterium (i.e., ²H) may afford certain therapeutic advantages resulting from greater metabolic stability, for example, increased in vivo half-life or reduced dosage requirements. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

In one general embodiment, the invention relates to N-pyridin-3-yl-4-{3-[4-(trifluoromethyl)phenoxy]benzyl}piperazine-1-carboxamide and pharmaceutically acceptable salts, pharmaceutically acceptable prodrugs, and pharmaceutically active metabolites of such a compound. In another general embodiment, the invention relates to pharmaceutical compositions each comprising a therapeutically effective amount of a FAAH-modulating agent selected from N-pyridin-3-yl-4-{3-[4-(trifluoromethyl)phenoxy]benzyl}piperazine-1-carboxamide and pharmaceutically acceptable salts, pharmaceutically acceptable prodrugs, and pharmaceutically active metabolites of such a compound.

The invention also relates to pharmaceutically acceptable salts of N-pyridin-3-yl-4-{3-[4-(trifluoromethyl)phenoxy]benzyl}piperazine-1-carboxamide. A “pharmaceutically acceptable salt” is intended to mean a salt of a free acid or base of a compound represented by Formula (I) that is non-toxic, biologically tolerable, or otherwise biologically suitable for administration to the subject. See, generally, G. S. Paulekuhn, et al., “Trends in Active Pharmaceutical Ingredient Salt Selection based on Analysis of the Orange Book Database”, J. Med. Chem., 2007, 50:6665-72, S. M. Berge, et al., “Pharmaceutical Salts”, J Pharm Sci., 1977, 66:1-19, and Handbook of Pharmaceutical Salts, Properties, Selection, and Use, Stahl and Wermuth, Eds., Wiley-VCH and VHCA, Zurich, 2002. Preferred pharmaceutically acceptable salts are those that are pharmacologically effective and suitable for contact with the tissues of patients without undue toxicity, irritation, or allergic response. Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methane-sulfonates, propanesulfonates, naphthalene-1-sulfonates, naphthalene-2-sulfonates, and mandelates.

In certain embodiments, the compound, N-pyridin-3-yl-4-{3-[4-(trifluoromethyl)phenoxy]benzyl}piperazine-1-carboxamide, is a hydrochloride salt. In further embodiments, the hydrochloride salt is bis-hydrochloride dihydrate.

A compound of N-pyridin-3-yl-4-{3-[4-(trifluoromethyl)phenoxy]benzyl}piperazine-1-carboxamide may possess a sufficiently basic group and accordingly may react with a number of inorganic and organic acids, to form a pharmaceutically acceptable salt.

The compound of the invention contains at least one basic nitrogen, therefore, a desired pharmaceutically acceptable salt may be prepared by any suitable method available in the art, for example, by treatment of the free base with an inorganic acid, such as hydrochloric acid, hydrobromic acid, sulfuric acid, sulfamic acid, nitric acid, boric acid, phosphoric acid, and the like; or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid; an amino acid, such as aspartic acid or glutamic acid; an aromatic acid, such as benzoic acid, 2-acetoxybenzoic acid, naphthoic acid, or cinnamic acid; a sulfonic acid, such as laurylsulfonic acid, p-toluenesulfonic acid, methanesulfonic acid, or ethanesulfonic acid; or any other acid and mixture thereof that are regarded as equivalents or acceptable substituents in light of the ordinary level of skill in this art.

The invention also relates to pharmaceutically acceptable prodrugs of N-pyridin-3-yl-4-{3-[4-(trifluoromethyl)phenoxy]benzyl}piperazine-1-carboxamide. The term “prodrug” means a precursor of a designated compound that, following administration to a subject, yields the compound in vivo via a chemical or physiological process such as solvolysis or enzymatic cleavage, or under physiological conditions (e.g., a prodrug on being brought to physiological pH is converted to the compound of Example 1). A “pharmaceutically acceptable prodrug” is a prodrug that is non-toxic, biologically tolerable, and otherwise biologically suitable for administration to the subject. Illustrative procedures for the selection and preparation of suitable prodrug derivatives are described, for example, in “Design of Prodrugs”, ed. H. Bundgaard, Elsevier, 1985.

Examples of prodrugs include compounds having an amino acid residue, or a polypeptide chain of two or more (e.g., two, three or four) amino acid residues, covalently joined through an amide or ester bond to a free amino group of N-pyridin-3-yl-4-{3-[4-(trifluoromethyl)phenoxy]benzyl}piperazine-1-carboxamide. Examples of amino acid residues include the twenty naturally occurring amino acids, commonly designated by three letter symbols, as well as 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone. Additional types of prodrugs may be produced, for instance, by derivatizing free amines as amides, sulfonamides or phosphonamides.

The present invention also relates to pharmaceutically active metabolites of N-pyridin-3-yl-4-{3-[4-(trifluoromethyl)phenoxy]benzyl}piperazine-1-carboxamide. A “pharmaceutically active metabolite” means a pharmacologically active product of metabolism in the body of N-pyridin-3-yl-4-{3-[4-(trifluoromethyl)phenoxy]benzyl}piperazine-1-carboxamide or a salt thereof. Prodrugs and active metabolites of a compound may be determined using routine techniques known or available in the art. See, e.g., Bertolini et al., J. Med. Chem. 1997, 40, 2011-2016; Shan et al., J. Pharm. Sci. 1997, 86 (7), 765-767; Bagshawe, Drug Dev. Res. 1995, 34, 220-230; Bodor, Adv. Drug Res. 1984, 13, 224-331; Bundgaard, Design of Prodrugs (Elsevier Press, 1985); and Larsen, Design and Application of Prodrugs, Drug Design and Development (Krogsgaard-Larsen et al., eds., Harwood Academic Publishers, 1991).

A compound of N-pyridin-3-yl-4-{3-[4-(trifluoromethyl)phenoxy]benzyl}piperazine-1-carboxamide, and its pharmaceutically acceptable salts, pharmaceutically acceptable prodrugs, and pharmaceutically active metabolites (collectively, “active agents”) of the present invention are useful as FAAH inhibitors in the methods of the invention. The term “inhibitors” refers to compounds that decrease, prevent, inactivate, desensitize or down-regulate FAAH expression or activity. The active agents may be used in the inventive methods for the treatment of medical conditions, diseases, or disorders mediated through inhibition or modulation of FAAH, such as those described herein. Active agents according to the invention may therefore be used as an analgesic, anti-depressant, cognition enhancer, neuroprotectant, sedative, appetite stimulant/suppressor, or contraceptive.

Exemplary medical conditions, diseases, and disorders mediated by FAAH activity include anxiety, depression, pain, sleep disorders, eating disorders, inflammation, multiple sclerosis and other movement disorders, HIV wasting syndrome, closed head injury, stroke, learning and memory disorders, Alzheimer's disease, epilepsy, Tourette's syndrome, epilepsy, Niemann-Pick disease, Parkinson's disease, Huntington's chorea, optic neuritis, autoimmune uveitis, symptoms of drug or alcohol withdrawal, nausea, emesis, sexual dysfunction, post-traumatic stress disorder, cerebral vasospasm, diabetes, metabolic syndrome, osteoarthritis and osteoporosis.

Thus, the active agents may be used to treat subjects diagnosed with or suffering from such a disease, disorder, or condition. The term “treat” or “treating” as used herein is intended to refer to administration of an agent or composition of the invention to a subject for the purpose of effecting a therapeutic benefit through inhibition of FAAH activity. Treating includes reversing, ameliorating, alleviating, inhibiting the progress of, lessening the severity of, reducing the incidence of, or preventing a disease, disorder, or condition, or one or more symptoms of such disease, disorder or condition mediated through modulation of FAAH activity. The term “subject” refers to a mammalian patient in need of such treatment, such as a human. “Modulators” include both inhibitors and activators, where “inhibitors” refer to compounds that decrease, prevent, inactivate, desensitize or down-regulate FAAH expression or activity, and “activators” are compounds that increase, activate, facilitate, sensitize, or up-regulate FAAH expression or activity.

Accordingly, the invention relates to methods of using the active agents described herein to treat subjects diagnosed with or suffering from a disease, disorder, or condition mediated through FAAH activity, such as: anxiety, pain, sleep disorders, eating disorders, inflammation, movement disorders (e.g., multiple sclerosis), glucose and lipid metabolism (e.g. diabetes) and bone homeostasis (e.g. osteoporosis).

Symptoms or disease states are intended to be included within the scope of “medical conditions, disorders, or diseases.” For example, pain may be associated with various diseases, disorders, or conditions, and may include various etiologies. Illustrative types of pain treatable with a FAAH-modulating agent, in one example herein a FAAH-inhibiting agent, according to the invention include cancer pain, postoperative pain, GI tract pain, spinal cord injury pain, visceral hyperalgesia, thalamic pain, headache (including stress headache and migraine), low back pain, neck pain, musculoskeletal pain, peripheral neuropathic pain, central neuropathic pain, neurogenerative disorder related pain, and menstrual pain. HIV wasting syndrome includes associated symptoms such as appetite loss and nausea. Parkinson's disease includes, for example, levodopa-induced dyskinesia. Treatment of multiple sclerosis may include treatment of symptoms such as spasticity, neurogenic pain, central pain, or bladder dysfunction. Symptoms of drug withdrawal may be caused by, for example, addiction to opiates or nicotine. Nausea or emesis may be due to chemotherapy, postoperative, or opioid related causes. Treatment of sexual dysfunction may include improving libido or delaying ejaculation. Treatment of cancer may include treatment of glioma. Sleep disorders include, for example, sleep apnea, insomnia, and disorders calling for treatment with an agent having a sedative or narcotic-type effect. Eating disorders include, for example, anorexia or appetite loss associated with a disease such as cancer or HIV infection/AIDS.

In treatment methods according to the invention, an effective amount of at least one active agent according to the invention is administered to a subject suffering from or diagnosed as having such a disease, disorder, or condition. A “therapeutically effective amount” or “effective amount” means an amount or dose of a FAAH-modulating agent sufficient to generally bring about a desired therapeutic benefit in patients in need of treatment for a disease, disorder, or condition mediated by FAAH activity. Effective amounts or doses of the active agents of the present invention may be ascertained by routine methods such as modeling, dose escalation studies or clinical trials, and by taking into consideration routine factors, e.g., the mode or route of administration or drug delivery, the pharmacokinetics of the agent, the severity and course of the disease, disorder, or condition, the subject's previous or ongoing therapy, the subject's health status and response to drugs, and the judgment of the treating physician. An exemplary dose is in the range of from about 0.0001 to about 200 mg of active agent per kg of subject's body weight per day, preferably about 0.001 to 100 mg/kg/day, or about 0.01 to 35 mg/kg/day, or about 0.1 to 10 mg/kg daily in single or divided dosage units (e.g., BID, TID, QID). For a 70-kg human, an illustrative range for a suitable dosage amount is from about 0.05 to about 7 g/day, or about 0.2 to about 5 g/day. Once improvement of the patient's disease, disorder, or condition has occurred, the dose may be adjusted for maintenance treatment. For example, the dosage or the frequency of administration, or both, may be reduced as a function of the symptoms, to a level at which the desired therapeutic effect is maintained. Of course, if symptoms have been alleviated to an appropriate level, treatment may cease. Patients may, however, require intermittent treatment on a long-term basis upon any recurrence of symptoms.

In addition, the active agents of the invention may be used in combination with additional active ingredients in the treatment of the above conditions. The additional active ingredients may be co-administered separately with an active agent of N-pyridin-3-yl-4-{3-[4-(trifluoromethyl)phenoxy]benzyl}piperazine-1-carboxamide, or included with such an agent in a pharmaceutical composition according to the invention. In an exemplary embodiment, additional active ingredients are those that are known or discovered to be effective in the treatment of conditions, disorders, or diseases mediated by FAAH activity, such as another FAAH modulator or a compound active against another target associated with the particular condition, disorder, or disease. The combination may serve to increase efficacy (e.g., by including in the combination a compound potentiating the potency or effectiveness of an active agent according to the invention), decrease one or more side effects, or decrease the required dose of the active agent according to the invention. In one illustrative embodiment, a composition according to the invention may contain one or more additional active ingredients selected from opioids, non-steroidal anti-inflammatory drugs (NSAID) (e.g., ibuprofen, cyclooxygenase-2 (COX-2) inhibitors, and naproxen), gabapentin, pregabalin, tramadol, acetaminophen, and aspirin.

The active agents of the invention are used, alone or in combination with one or more additional active ingredients, to formulate pharmaceutical compositions of the invention. A pharmaceutical composition of the invention comprises: (a) an effective amount of at least one active agent in accordance with the invention; and (b) a pharmaceutically acceptable excipient.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of a agent and that is compatible therewith. Examples of excipients include calcium carbonate, calcium phosphate, various sugars and types of starch, cellulose derivatives, gelatin, vegetable oils, and polyethylene glycols.

Delivery forms of the pharmaceutical compositions containing one or more dosage units of the active agents may be prepared using suitable pharmaceutical excipients and compounding techniques known or that become available to those skilled in the art. The compositions may be administered in the inventive methods by a suitable route of delivery, e.g., oral, parenteral, rectal, topical, or ocular routes, or by inhalation.

The preparation may be in the form of tablets, capsules, sachets, dragees, powders, granules, lozenges, powders for reconstitution, liquid preparations, or suppositories. Preferably, the compositions are formulated for intravenous infusion, topical administration, or oral administration.

For oral administration, the active agents of the invention can be provided in the form of tablets or capsules, or as a solution, emulsion, or suspension. To prepare the oral compositions, the active agents may be formulated to yield a dosage of, e.g., from about 5 mg to 5 g daily, or from about 50 mg to 5 g daily, in single or divided doses. For example, a total daily dosage of about 5 mg to 5 g daily may be accomplished by dosing once, twice, three, or four times per day.

Oral tablets may include the active ingredient(s) mixed with compatible pharmaceutically acceptable excipients such as diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are exemplary disintegrating agents. Binding agents may include starch and gelatin. The lubricating agent, if present, may be magnesium stearate, stearic acid or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, active ingredient(s) may be mixed with a solid, semi-solid, or liquid diluent. Soft gelatin capsules may be prepared by mixing the active ingredient with water, an oil such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions or syrups or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The active agents of this invention may also be administered by non-oral routes. For example, compositions may be formulated for rectal administration as a suppository. For parenteral use, including intravenous, intramuscular, intraperitoneal, or subcutaneous routes, the agents of the invention may be provided in sterile aqueous solutions or suspensions, buffered to an appropriate pH and isotonicity or in parenterally acceptable oil. Suitable aqueous vehicles include Ringer's solution and isotonic sodium chloride. Such forms may be presented in unit-dose form such as ampules or disposable injection devices, in multi-dose forms such as vials from which the appropriate dose may be withdrawn, or in a solid form or pre-concentrate that can be used to prepare an injectable formulation. Illustrative infusion doses range from about 1 to 1000 μg/kg/minute of agent admixed with a pharmaceutical carrier over a period ranging from several minutes to several days.

For topical administration, the agents may be mixed with a pharmaceutical carrier at a concentration of about 0.1% to about 10% of drug to vehicle. Another mode of administering the agents of the invention may utilize a patch formulation to affect transdermal delivery.

Active agents may alternatively be administered in methods of this invention by inhalation, via the nasal or oral routes, e.g., in a spray formulation also containing a suitable carrier.

Exemplary active agents useful in methods of the invention will now be described by reference to illustrative synthetic schemes for their general preparation below and the specific examples that follow.

The compounds as described above may be made according to processes within the skill of the art and/or that are described in the schemes and examples that follow. Certain reaction schemes may occur with or without protection as appropriate. This may be achieved by means of conventional protecting groups, such as those described in “Protective Groups in Organic Chemistry”, ed. J. F. W. McOmie, Plenum Press, 1973; and T. W. Greene & P. G. M. Wuts, “Protective Groups in Organic Synthesis”, 3^{rd} ed., John Wiley & Sons, 1999. The protecting groups may be removed at a convenient subsequent stage using methods known from the art. Alternatively, it may be necessary to employ, in the place of the ultimately desired substituent, a suitable group that may be carried through the reaction scheme and replaced as appropriate with the desired substituent. Such compounds, precursors, or prodrugs are also within the scope of the invention.

In order to illustrate the invention, the following examples are included. These examples do not limit the invention. They are only meant to suggest a method of practicing the invention. Those skilled in the art may find other methods of practicing the invention, which are obvious to them. However, those methods are deemed to be within the scope of this invention.

### CHEMICAL EXAMPLES

The compound of the invention is made according to the following Example 1.

Reaction mixtures were stirred under a nitrogen atmosphere unless otherwise noted. Where solutions or mixtures are concentrated, they are typically concentrated under reduced pressure using a rotary evaporator.

Mass spectra were obtained on an Agilent series 1100 MSD using electrospray ionization (ESI) in positive mode unless otherwise indicated.

NMR spectra were obtained on either a Bruker model DPX400 (400 MHz), DPX500 (500 MHz), DRX600 (600 MHz) spectrometer. The format of the ¹H NMR data below is: chemical shift in ppm down field of the tetramethylsilane reference (multiplicity, coupling constant J in Hz, integration).

Chemical names were generated using Chem Draw Ultra 6.0.2 (CambridgeSoft Corp., Cambridge, Mass.) or ACD/Name Version 9 (Advanced Chemistry Development, Toronto, Ontario, Canada).

### Intermediate 1: Pyridin-3-yl-carbamic acid phenyl ester

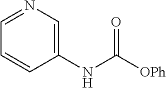

To a solution consisting of 9.49 g (101 mmol) pyridin-3-yl amine and 8.77 g (111 mmol) pyridine in 80 mL CH₃CN at 0° C. was added 15.8 g (101 mmol) phenyl chloroformate dropwise. The reaction mixture was allowed to warm to room temperature and stirred for 2 h. The reaction was quenched with H₂O (200 mL) and the resulting precipitate was filtered and dried under vacuum to provide the title compound as a tan solid (17.34 g, 80%). MS (ESI⁺): calcd for C₁₂H₁₀N₂O₂ m/z 214.07, found 215.3 (M+H)⁺. ¹H NMR (500 MHz, d₆-DMSO): 10.46 (s, 1H), 8.69 (d, J=2.4 Hz, 1H), 8.27 (dd, J=4.7, 1.4 Hz, 1H), 7.93 (d, J=8.4 Hz, 1H), 7.47-7.41 (m, 2H), 7.37 (dd, J=8.4, 4.7 Hz, 1H), 7.31-7.22 (m, 3H).

### Intermediate 2: Piperazine-1-carboxylic acid pyridin-3-ylamide, bis-hydrochloride salt

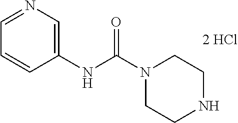

A 3-L round-bottomed flask equipped with a mechanical stirrer, an addition funnel, a gas inlet, a heating mantle, and a thermocouple probe was charged with 150 g (700 mmol) Intermediate 1, 124 g (667 mmol) 1-Boc-piperazine, and 1.1 L MeOH. The reaction was heated to 50° C., under N₂, for 20 hours. The reaction was then cooled to 25° C. and then opened to air. Concentrated HCl (250 mL, 3000 mmol) was then added dropwise over 75 minutes. The reaction was heated to 50° C., stirred for 1 hour, and then cooled in an ice bath for 3 h. The precipitate was collected via filtration, washed with 150 mL cold MeOH, and placed in a drying oven (ca. 25° C., −26 in. Hg, N₂ purge, 72 h) to afford piperazine-1-carboxylic acid pyridin-3-ylamide dihydrochloride as a tan solid (157.3 g, 84% yield). ¹H NMR (400 MHz, CDCl₃): 10.45 (s, 1H), 9.58 (s, 2H), 9.16 (d, J=2.3, 1H), 8.75-8.65 (m, 1H), 8.51 (d, J=5.4, 1H), 7.95 (dd, J=8.7, 5.5, 1H), 3.91-3.77 (m, 4H), 3.14 (s, 4H); MS (ESI⁺) Calcd for C₁₀H₁₅N₄O [M+H]⁺, 207.1; m/z, found 207.0.

### Example 1

### N-Pyridin-3-yl-4-{3-[4-(trifluoromethyl)phenoxy]benzyl}piperazine-1-carboxamide, bis-hydrochloride dihydrate

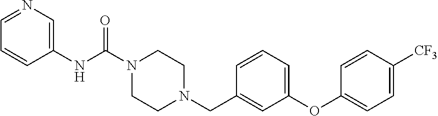

N-Pyridin-3-yl-4-{3-[4-(trifluoromethyl)phenoxy]benzyl}piperazine-1-carboxamide: a 2-L round-bottomed flask equipped with a mechanical stirrer, a reflux condenser, a gas inlet, a heating mantle, and a thermocouple probe was charged with 43.6 g (357 mmol) 3-hydroxybenzaldehyde, 50.0 mL (357 mmol) 4-bromobenzotrifluoride, 2.24 g Cul (11.8 mmol), 5.0 g (35.7 mmol) N,N-dimethylglycine hydrochloride, 98.7 g (714 mmol) potassium carbonate, and 446 mL DMF. With mechanical stirring, the reaction was heated to 120° C. under N₂ for 24 hours, and then cooled to 25° C. The suspension was filtered and the resulting solid rinsed with 250 mL DMF. The combined filtrates were placed in a flask to which was added 94.7 g (339 mmol) Intermediate 2. The flask was placed in a room temperature water bath and then charged with 79.1 g (373 mmol) sodium triacetoxyborohydride portion-wise over 15 minutes. The reaction was stirred at ambient temperature for 18 hours. The reaction mixture was poured into water and the pH adjusted to 8 with 50% NaOH. The product was extracted with MTBE (841 mL), dried over Na₂SO₄, and concentrated to give crude 4-[3-(4-trifluoromethyl-phenoxy)-benzyl]-piperazine-1-carboxylic acid pyridin-3-ylamide as a thick black oil (183 g, 107% crude yield). The crude material was used in the next step without further purification.

N-Pyridin-3-yl-4-{3-[4-(trifluoromethyl) phenoxy]benzyl}piperazine-1-carboxamide bis-hydrochloride dihydrate: a 3 L, three-neck Morton flask equipped with a mechanical stirrer, thermocouple, and addition funnel was charged with 155 g (339 mmol) crude N-pyridin-3-yl-4-{3-[4-(trifluoromethyl)phenoxy]benzyl}piperazine-1-carboxamide, 1.41 L acetone, and 28 mL water. The resultant dark solution was heated to 45° C. The heating mantle was removed and concentrated aqueous HCl (˜12 M, 56.5 mL) was added over 10 min. The solution was slowly cooled to room temperature and then placed in an ice bath for 1 h. The solid formed was collected by filtration, rinsed with acetone, and dried on a vacuum funnel to give product 4-[3-(4-trifluoromethyl-phenoxy)-benzyl]-piperazine-1-carboxylic acid pyridin-3-ylamide bis-hydrochloride dihydrate as a light pink solid (95 g, 50%). MS (ESI⁺): Calcd for C₂₄H₂₄F₃N₄O₂ [M+H]⁺, 457.2; m/z found 457.1; Anal. Calcd for C₂₄H₂₉Cl₂F₃N₄O₄: C, 50.98; H, 5.17; N, 9.91. Found: C, 51.22; H, 4.96; N, 9.93; ¹H NMR (400 MHz, d₆-DMSO): 11.76 (s, 1H), 10.54 (s, 1H), 9.17 (d, J=2.3 Hz, 1H), 8.72 (ddd, J=8.7, 2.3, 1.1 Hz, 1H), 8.53 (d, J=5.4 Hz, 1H), 7.97 (dd, J=8.7, 5.5 Hz, 1H), 7.77 (d, J=8.6 Hz, 2H), 7.61-7.49 (m, 2H), 7.47 (m, 1H), 7.31-7.16 (m, 3H), 4.43 (br s, 2H), 4.38 (s, 2H), 3.41 (br s, 4H), 3.08 (br s, 2H); ¹³C NMR (101 MHz, d₆-DMSO): 160.22, 155.56, 154.17, 140.88, 134.71, 134.56, 132.17, 131.62, 131.18, 128.07, 127.93 (q, J_{C-F}=3.6 Hz), 127.42, 124.62 (q, J_{C-F}=272.3 Hz), 124.00 (q, J_{C-F}=32.6 Hz), 122.99, 121.21, 118.71, 58.30, 50.59, 41.29.

### Biological Testing

### Assay Method 1

A. Transfection of Cells with Human FAAH

A 10-cm tissue culture dish with a confluent monolayer of SK-N-MC cells was split 2 days (d) prior to transfection. Using sterile technique, the media was removed and the cells were detached from the dish by the addition of trypsin. One fifth of the cells were then placed onto a new 10-cm dish. Cells were grown in a 37° C. incubator with 5% CO₂ in Minimal Essential Media Eagle with 10% Fetal Bovine Serum. After 2 d, cells were approximately 80% confluent. These cells were removed from the dish with trypsin and pelleted in a clinical centrifuge. The pellet was re-suspended in 400 μL complete media and transferred to an electroporation cuvette with a 0.4 cm gap between the electrodes. Supercoiled human FAAH cDNA (1 μg), corresponding to NCBI accession NM_{—}001441, was added to the cells and mixed. The voltage for the electroporation was set at 0.25 kV, and the capacitance was set at 960 μF. After electroporation, the cells were diluted into complete media (10 mL) and plated onto four 10-cm dishes. Due to variability in the efficiency of electroporation, four different concentrations of cells were plated. The ratios used were 1:20, 1:10, and 1:5, with the remainder of the cells being added to the fourth dish. The cells were allowed to recover for 24 h before adding the selection media (complete media with 600 μg/mL G418). After 10 d, dishes were analyzed for surviving colonies of cells. Dishes with well-isolated colonies were used. Cells from individual colonies were isolated and tested. The clones that showed the most FAAH activity, as measured by anandamide hydrolysis, were used for further study.

B. FAAH Assay

T84 frozen cell pellets or transfected SK-N-MC cells (contents of 1×15 cm culture dishes) were homogenized in 50 mL of FAAH assay buffer (125 mM Tris, 1 mM EDTA, 0.2% Glycerol, 0.02% Triton X-100, 0.4 mM Hepes, pH 9). The assay mixture consisted of 50 μL of the cell homogenate, 10 μL of the test compound, and 40 μL of anandamide [1-³H-ethanolamine] (³H-AEA, Perkin-Elmer, 10.3 Cᵢ/mmol), which was added last, for a final tracer concentration of 80 nM. The reaction mixture was incubated at rt for 1 h. During the incubation, 96-well Multiscreen filter plates (catalog #MAFCNOB50; Millipore, Bedford, Mass., USA) were loaded with 25 μL of activated charcoal (Multiscreen column loader, catalog #MACL09625, Millipore) and washed once with 100 μL of MeOH. Also during the incubation, 96-well DYNEX MicroLite plates (catalog #NL510410) were loaded with 100 μL of MicroScint40 (catalog #6013641, Packard Bioscience, Meriden, Conn., USA). After the 1 h incubation, 60 μL of the reaction mixture were transferred to the charcoal plates, which were then assembled on top of the DYNEX plates using Centrifuge Alignment Frames (catalog #MACF09604, Millipore). The unbound labeled ethanolamine was centrifuged through to the bottom plate (5 min at 2000 rpm), which was preloaded with the scintillant, as described above. The plates were sealed and left at rt for 1 h before counting on a Hewlett Packard TopCount.

### Assay Method 2

A. Transfection of Cells with Rat FAAH

A 10-cm tissue culture dish with a confluent monolayer of SK-N-MC cells was split 2 d prior to transfection. Using sterile technique, the media was removed and the cells were detached from the dish by the addition of trypsin. One fifth of the cells were then placed onto a new 10-cm dish. Cells were grown in a 37° C. incubator with 5% CO₂ in Minimal Essential Media Eagle containing 10% Fetal Bovine Serum. After 2 d, cells were approximately 80% confluent. These cells were removed from the dish with trypsin and pelleted in a clinical centrifuge. The pellet was re-suspended in 400 μL complete media and transferred to an electroporation cuvette with a 0.4 cm gap between the electrodes. Supercoiled rat FAAH cDNA (1 μg), corresponding to NCBI accession NM_{—}024132, was added to the cells and mixed. The voltage for the electroporation was set at 0.25 kV, and the capacitance was set at 960 μF. After electroporation, the cells were diluted into complete media (10 mL) and plated onto four 10-cm dishes. Because of the variability in the efficiency of electroporation, four different concentrations of cells were plated. The ratios used were 1:20, 1:10, and 1:5, with the remainder of the cells being added to the fourth dish. The cells were allowed to recover for 24 h before adding the selection media (complete media with 600 μg/mL G418). After 10 d, dishes were analyzed for surviving colonies of cells. Dishes with well-isolated colonies were used. Cells from individual colonies were isolated and tested. The clones that showed the most FAAH activity, as measured by anandamide hydrolysis, were used for further study.

B. FAAH Assay

T84 frozen cell pellets or transfected SK-N-MC cells (contents of 1×15 cm culture dishes) were homogenized in 50 mL of FAAH assay buffer (125 mM Tris, 1 mM EDTA, 0.2% Glycerol, 0.02% Triton X-100, 0.4 mM Hepes, pH 9). The assay mixture consisted of 50 μL of the cell homogenate, 10 μL of the test compound, and 40 μL of anandamide [1-³H-ethanolamine] (³H-AEA, Perkin-Elmer, 10.3 Cᵢ/mmol), which was added last, for a final tracer concentration of 80 nM. The reaction mixture was incubated at rt for 1 h. During the incubation, 96-well Multiscreen filter plates (catalog #MAFCNOB50; Millipore, Bedford, Mass., USA) were loaded with 25 μL of activated charcoal (Multiscreen column loader, catalog #MACL09625, Millipore) and washed once with 100 μL of MeOH. Also during the incubation, 96-well DYNEX MicroLite plates (catalog #NL510410) were loaded with 100 μL of MicroScint40 (catalog #6013641, Packard Bioscience, Meriden, Conn., USA). After the 1 h incubation, 60 μL of the reaction mixture were transferred to the charcoal plates, which were then assembled on top of the DYNEX plates using Centrifuge Alignment Frames (catalog #MACF09604, Millipore). The unbound labeled ethanolamine was centrifuged through to the bottom plate (5 min at 2000 rpm), which was preloaded with the scintillant, as described above. The plates were sealed and left at rt for 1 h before counting on a Hewlett Packard TopCount. Results for compounds tested in these assays are summarized in Table 1 as an average value and the standard error of the mean reported in parenthesis. N-Pyridin-3-yl-4-{3-[4-(trifluoromethyl)phenoxy]benzyl}piperazine-1-carboxamide was tested as either a free base or the HCl salt form. Comparator compounds 4-[3-(4-bromophenoxy)benzyl]-N-pyridin-3-ylpiperazine-1-carboxamide and 4-({3-[(4-chlorophenyl)oxy]phenyl}methyl)-N-pyridin-3-ylpiperazine-1-carboxamide were prepared using methods and materials analogous to those described in WO 2006/074025. 4-({3-[(4-Chlorophenyl)oxy]phenyl}methyl)-N-pyridin-3-ylpiperazine-1-carboxamide was tested as either a trifluoroacetic acid salt or HCl salt form. 4-[3-(4-Bromophenoxy)benzyl]-N-pyridin-3-ylpiperazine-1-carboxamide was tested as tested as either a free base or the HCl salt form.

TABLE 1

| | Assay 1 | Assay 2 |
|---|---|---|
| Sample | IC₅₀ (nM) | IC₅₀ (nM) |
| Example 1 | 1.0 (0.5) | 18 (3.5) |
| 4-[3-(4-Bromophenoxy)benzyl]-N-pyridin-3- | 1.0 (0.4) | 22 (6.6) |
| ylpiperazine-1-carboxamide | | |
| 4-({3-[(4-Chlorophenyl)oxy]phenyl}methyl)- | 2.4 (0.8) | 65 (21) |
| N-pyridin-3-ylpiperazine-1-carboxamide | | |

N-Pyridin-3-yl-4-{3-[4-(trifluoromethyl)phenoxy]benzyl}piperazine-1-carboxamide exhibited potent activity as an inhibitor of anandamide hydrolysis by human and rat FAAH. Comparator compounds 4-[3-(4-bromophenoxy)benzyl]-N-pyridin-3-ylpiperazine-1-carboxamide and 4-({3-[(4-chlorophenyl)oxy]phenyl}methyl)-N-pyridin-3-ylpiperazine-1-carboxamide showed similar potency to the human and rat FAAH.

Assay Method 3

Langendorff Isolated Heart Protocol

Male Sprague-Dawley rats (300-500 g) were anesthetized by an intraperitoneal injection of sodium pentobarbital (50 mg/kg), and then euthanized via cardiectomy. Hearts were rapidly removed, mounted on a

Langendorff apparatus, and perfused with modified, oxygenated, recirculating Krebs-Henseleit buffer (118.1 mM NaCl, 4.7 mM KCl, 1.17 mM MgSO₄, 1.18 mM KH₂PO₄, 11.1 mM d-glucose, 2.5 mM CaCl₂, 24.8 mM NaHCO₃, 2.0 mM pyruvate, and 1.25 g\L BSA in deionized, reagent grade water). A ventricular drain and fluid-filled latex balloon was secured in the left ventricle with a purse string suture at the atrial appendage. Hearts were not paced. Hearts were deemed acceptable for use in the study if they exhibit acceptable hemodynamic (left ventricular developed pressure >40 mmHg, and coronary perfusion pressure 60-90 mmHg) parameters throughout the equilibration period. The hearts were maintained at approximately 37° C. while suspended in a glass chamber and bathed with Krebs-Henseleit buffer. The latex balloon in the left ventricle (LV) was expanded with water to achieve a LV end-diastolic pressure (LVEDP) of approximately 10-15 mmHg. The balloon was connected via tubing to a pressure transducer to measure LVEDP, LV diastolic pressure (LVDP) and LV systolic pressure (LVSP). Coronary perfusion pressure (CPP) was measured with a pressure transducer connected to a side-arm port of the aortic cannula.

Individual hearts were equilibrated for approximately 10-15 minutes. Following this equilibration period, baseline measurements were collected for approximately 15 minutes. After this period, a first heart was exposed to test compound for a total 75 min (comprising five increasing concentrations of test compound for approximately 15 minutes at each concentration) with measurements collected continuously. Test compound was dissolved in DMSO and then added directly to the experiment perfusion medium. Based on these results, four additional hearts were subsequently exposed to test compound for a total of 60 min (comprising four increasing concentrations of test compound for approximately 15 minutes at each concentration), after an initial 15 min equilibration period, with measurements collected continuously.

A one mL sample of perfusion medium was taken from the heart exudate at the end of each 15 minute monitoring period. The samples were frozen on dry ice, stored in a freezer set to maintain −80° C. until analysis. At the conclusion of the experiment, the hearts were frozen with liquid nitrogen and stored in a freezer set to maintain a temperature of −80° C. until analyzed.

### Results

Comparator compound 4-({3-[(4-chlorophenyl)oxy]phenyl}methyl)-N-pyridin-3-ylpiperazine-1-carboxamide bis-hydrochloride mono-hydrate caused a marked increase in left ventricular systolic pressure (LVSP) at the maximum concentration tested (30 μM). In contrast, N-pyridin-3-yl-4-{3-[4-(trifluoromethyl)phenoxy]benzyl}piperazine-1-carboxamide bis-hydrochloride dihydrate did not induce an abrupt increase in LVSP at a maximum concentration of 30 μM. In addition, the compounds differed in their effects on left ventricular developed pressure (LVDevP). Whereas 4-({3-[4-chlorophenyl)oxy]phenyl}methyl)-N-pyridin-3-ylpiperazine-1-carboxamide bis-hydrochloride mono-hydrate at 30 μM resulted in a near-doubling of LVDevP, N-Pyridin-3-yl-4-{3-[4-(trifluoromethyl) phenoxy]benzyl}piperazine-1-carboxamide bis-hydrochloride dihydrate resulted in a decrease in LVDevP. Furthermore, there was a notable difference in the effects of the two compounds on heart rate. Whereas all hearts (n=4) perfused with 4-({3-[(4-chlorophenyl)oxy]phenyl}methyl)-N-pyridin-3-ylpiperazine-1-carboxamide bis-hydrochloride mono-hydrate at 30 μM showed profound bradycardia characterized by a greater than 30% slowing of heart rate, only two of five hearts perfused at the same concentration with N-pyridin-3-yl-4-{3-[4-(trifluoromethyl)phenoxy]benzyl}piperazine-1-carboxamide bis-hydrochloride dihydrate showed greater than a 30% decrease in heart rate. The observations between these two compounds are surprising due to the molecular structures and difference in enzymatic potencies of the two compounds. N-pyridin-3-yl-4-{3-[4-(trifluoromethyl)phenoxy]benzyl}piperazine-1-carboxamide is about 3.5 times more potent in in vitro assays using rat FAAH enzyme (see Assay Method 1 above).

Evaluation of comparator compound 4-[3-(4-bromophenoxy)benzyl]-N-pyridin-3-ylpiperazine-1-carboxamide bis-hydrochloride mono-hydrate with N-pyridin-3-yl-4-{3-[4-(trifluoromethyl) phenoxy]benzyl}piperazine-1-carboxamide bis-hydrochloride dihydrate further exhibited an unpredictable favorable differentiation with respect to heart rate. Whereas three of four hearts perfused with a bath concentration of 26 μM of 4-[3-(4-bromophenoxy)benzyl]-N-pyridin-3-ylpiperazine-1-carboxamide bis-hydrochloride mono-hydrate displayed greater than 30% heart rate slowing, only two of five hearts perfused with 30 μM of N-pyridin-3-yl-4-{3-[4-(trifluoromethyl)phenoxy]benzyl}piperazine-1-carboxamide bis-hydrochloride dihydrate showed greater than 30% slowing of heart rate. These observations are unexpected due to the molecular structures and in that both compounds show similar potencies in the rat FAAH enzyme assay (see Assay Method 1 above).

### Assay Method 4

Rat Screening Toxicity Protocol

Male Crl:CD® (SD) rats approximately 8 weeks of age and weighing approximately 200 to 350 grams at initiation of dosing, were supplied by Charles River Laboratories, Hollister, Calif. Rats were individually housed in solid bottom plastic cages with 0.25 inch Cobb Bedding. Study rooms were maintained on a 12-hour light/dark cycle, with a temperature range of 64 to 79° F., and a relative humidity range of 30 to 70%. Rats were fed ad libitum (except where noted) with Rodent Diet 5008 supplied by PMI® Nutrition International, Richmond, Ind. Water was provided ad libitum by an automatic watering system and/or water bottles. The animals were quarantined for at least 5 days (prior to treatment period). Rats were selected on the basis of predose evaluations and randomly assigned to groups using a computer-generated randomization method based on body weight.

A vehicle treated group was dosed to match the highest dose volume (mL/kg) used. The test compound was administered orally daily. Test compounds were stored at room temperature. 4-({3-[(4-chlorophenyl)oxy]phenyl}methyl)-N-pyridin-3-ylpiperazine-1-carboxamide was dosed in solution using 20% cyclodextrin as the vehicle. N-pyridin-3-yl-4-{3-[4-(trifluoromethyl)phenoxy]benzyl}piperazine-1-carboxamide bis-hydrochloride dihydrate was dosed in solution using water as the vehicle. Rats in the vehicle treated and test article treated were dosed for up to 4 days. Mortality checks were performed at least once a day on all animals pre-dose and post-dose. Clinical observations were performed at least once during the pre-dose period. Clinical observations were recorded prior to dosing, at least once after daily dosing, and prior to scheduled necropsy for animals assessed for toxicology. Body Weights were determined at least once during the pre-dose period, prior to the first dose, once during dose administration and once after the last dose, and once prior to necropsy for tissue body weight ratios. On Day 4, rats designated for toxicology were fasted overnight. On Day 5 they were anesthetized using isoflurane, euthanized by exsanguination, and necropsied. The thoracic and abdominal cavities were examined and heart tissue was fixed in 10% neutral buffered formalin prior to microscopic examination. Observations were recorded at necropsy and tissue trimming. Any rats/groups that were removed from the study and rats that died prior to scheduled necropsy were necropsied. Clinical pathology was not determined on these animals.

Histology slides of heart tissue were prepared for all Toxicology animals. A section of heart was taken at scheduled necropsy and immediately frozen in liquid nitrogen. Tissues in fixative were processed into slides stained with hematoxylin and eosin.

### Results

Rats treated daily with 300 mg/kg of comparator compound 4-({3-[(4-chlorophenyl)oxy]phenyl}methyl)-N-pyridin-3-ylpiperazine-1-carboxamide bis-hydrochloride mono-hydrate developed lesions in heart muscle, whereas such tissue damage was absent in animals treated with N-pyridin-3-yl-4-{3-[4-(trifluoromethyl)phenoxy]benzyl}piperazine-1-carboxamide bis-hydrochloride dihydrate at the same dose. These observations are unexpected given that N-pyridin-3-yl-4-{3-[4-(trifluoromethyl) phenoxy]benzyl}piperazine-1-carboxamide is about 3.5 times more potent in in vitro assays using rat FAAH enzyme (see Assay Method 1 above).

While the invention has been illustrated by reference to exemplary and preferred embodiments, it will be understood that the invention is intended not to be limited to the foregoing detailed description, but to be defined by the appended claims as properly construed under principles of patent law.

What is claimed is:

1. A compound that is N-pyridin-3-yl-4-{3-[4-(trifluoromethyl)phenoxy]benzyl}piperazine-1-carboxamide or a pharmaceutically acceptable salt thereof.

2. The pharmaceutically acceptable salt of claim 1, wherein said salt is a hydrochloride salt of N-pyridin-3-yl-4-{3-[4-(trifluoromethyl)phenoxy]benzyl}piperazine-1-carboxamide.

3. The pharmaceutically acceptable salt of claim 2, wherein said hydrochloride salt is bis-hydrochloride dihydrate.

4. A pharmaceutical composition comprising:
(a) a therapeutically effective amount of N-pyridin-3-yl-4-{3-[4-(trifluoromethyl)phenoxy]benzyl}piperazine-1-carboxamide and a pharmaceutically acceptable salt thereof; and
(b) a pharmaceutically acceptable excipient.

5. A method for modulating FAAH activity, comprising exposing FAAH to a therapeutically effective amount of at least one of N-pyridin-3-yl-4-{3-[4-(trifluoromethyl)phenoxy]benzyl}piperazine-1-carboxamide and a pharmaceutically acceptable salt thereof.

6. A method of treating a subject suffering from or diagnosed with a disease, disorder, or medical condition mediated by FAAH activity, comprising administering to the subject in need of such treatment a therapeutically effective amount of a compound as defined in claim 5.

7. A method according to claim 6, wherein the disease, disorder, or medical condition is selected from the group consisting of: anxiety, depression, pain, sleep disorders, eating disorders, inflammation, movement disorders, HIV wasting syndrome, closed head injury, stroke, learning and memory disorders, Alzheimer's disease, epilepsy, Tourette's syndrome, Niemann-Pick disease, Parkinson's disease, Huntington's chorea, optic neuritis, autoimmune uveitis, drug withdrawal, nausea, emesis, sexual dysfunction, post-traumatic stress disorder, cerebral vasospasm, glaucoma, irritable bowel syndrome, inflammatory bowel disease, immunosuppression, gastroesophageal reflux disease, paralytic ileus, secretory diarrhea, gastric ulcer, rheumatoid arthritis, unwanted pregnancy, hypertension, cancer, hepatitis, allergic airway disease, autoimmune diabetes, intractable pruritis, neuroinflammation, diabetes, metabolic syndrome, and osteoporosis.

8. A method according to claim 6, wherein the disease, disorder, or medical condition is pain or inflammation.

9. A method according to claim 6, wherein the disease, disorder, or medical condition is anxiety, a sleep disorder, an eating disorder, or a movement disorder.

10. A method according to claim 6, wherein the disease, disorder, or medical condition is multiple sclerosis.

11. A method according to claim 6, wherein the disease, disorder, or medical condition is energy metabolism or bone homeostasis.
